(19) United States
(12) Patent Application Publication
(10) Pub. No.: US 2011/0218217 A1
(43) Pub. Date: Sep. 8, 2011

(54) PIPERIDYLPROPIONAMIDE DERIVATIVES USEFUL FOR THE TREATMENT OF CNS DISORDERS INCLUDING DEPRESSION AND PANIC DISORDER

(75) Inventors: Dan Peters, Malmo (SE); John Paul Redrobe, Rodovre (DK); Elsebet Østergaard Nielsen, Kobenhavn K (DK)

(73) Assignee: NEUROSEARCH A/S, Ballerup (DK)

(21) Appl. No.: 13/061,353

(22) PCT Filed: Aug. 25, 2009
(86) PCT No.: PCT/EP2009/060909

§ 371 Date: May 17, 2011

Related U.S. Application Data
(62) Division of application No. 61/093,765, filed on Sep. 3, 2008.

(30) Foreign Application Priority Data
Sep. 1, 2008 (DK) PA 2008 01210

(51) Int. Cl. A61K 31/445; C07D 211/34; A61K 31/4465; A61P 25/00; A61P 25/24; A61P 25/22; A61P 3/04; A61P 25/16; A61P 25/28; A61P 25/30; A61P 25/34; A61P 25/32; A61P 29/00; A61P 19/02; A61P 25/06; A61P 1/00

(52) U.S. Cl. 514/319; 546/233; 546/230; 546/205; 546/206; 514/331

(57) ABSTRACT

This invention relates to novel piperidine-4-propionamide derivatives useful as monoamine neurotransmitter re-uptake inhibitors. In other aspects the invention relates to the use of these compounds in a method for therapy and to pharmaceutical compositions comprising the compounds of the invention.

### TECHNICAL FIELD

This invention relates to novel piperidine-4-propionamide derivatives useful as monoamine neurotransmitter re-uptake inhibitors.

In other aspects the invention relates to the use of these compounds in a method for therapy and to pharmaceutical compositions comprising the compounds of the invention.

### BACKGROUND ART

Serotonin Selective Reuptake Inhibitors (SSRIs) currently provide efficacy in the treatment of several CNS disorders, including depression and panic disorder. SSRIs are generally perceived by psychiatrists and primary care physicians as effective, well-tolerated and easily administered. However, they are associated with a number of undesirable features.

Thus, there is still a strong need for compounds with an optimised pharmacological profile as regards the activity on reuptake of the monoamine neurotransmitters serotonin, dopamine and noradrenaline, such as the ratio of the serotonin reuptake versus the noradrenaline and dopamine reuptake activity.

### SUMMARY OF THE INVENTION

It is an object of the invention to provide novel compounds which show activity as monoamine neurotransmitter re-uptake inhibitors.

In one aspect, the invention provides a compound of Formula (I):

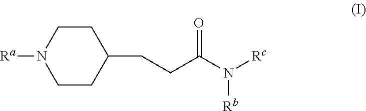

any of its stereoisomers or any mixture of its stereoisomers, or a pharmaceutically acceptable salt thereof; wherein R^{a}, R^{b} and R^{c} are as defined below.

In another aspect, the invention provides a pharmaceutical composition, comprising a therapeutically effective amount of a compound of the invention, any of its stereoisomers or any mixture of its stereoisomers, or a pharmaceutically acceptable salt thereof, together with at least one pharmaceutically acceptable carrier, excipient or diluent.

In another aspect, the invention provides the use of a compound of the invention, any of its stereoisomers or any mixture of its stereoisomers, or a pharmaceutically acceptable salt thereof, for the manufacture of a pharmaceutical composition for the treatment, prevention or alleviation of a disease or a disorder or a condition of a mammal, including a human, which disease, disorder or condition is responsive to inhibition of monoamine neurotransmitter re-uptake in the central nervous system.

In another aspect, the invention relates to a method for treatment, prevention or alleviation of a disease or a disorder or a condition of a living animal body, including a human, which disorder, disease or condition is responsive to responsive to inhibition of monoamine neurotransmitter re-uptake in the central nervous system, which method comprises the step of administering to such a living animal body in need thereof a therapeutically effective amount of a compound of the invention, any of its stereoisomers or any mixture of its stereoisomers, or a pharmaceutically acceptable salt thereof.

Other objects of the invention will be apparent to the person skilled in the art from the following detailed description and examples.

### DETAILED DISCLOSURE OF THE INVENTION

In one aspect the present invention provides compounds of Formula (I):

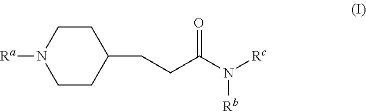

any of its stereoisomers or any mixture of its stereoisomers, or a pharmaceutically acceptable salt thereof, wherein
R^{a} and R^{b}, independently of each other, represent hydrogen or C₁₋₆-alkyl;
R^{c} represents phenyl or naphthyl, which phenyl and naphtyl are optionally substituted with one or more substituents independently selected from the group consisting of halo, trifluoromethyl, trifluoromethoxy, cyano and alkoxy.

In one embodiment of the invention, in formula (I), R^{a} represents hydrogen. In another embodiment, R^{a} represents C₁₋₆-alkyl, e.g. methyl or ethyl.

In another embodiment of the invention, in formula (I), R^{b} represents hydrogen. In another embodiment, R^{b} represents C₁₋₆-alkyl, e.g. methyl, ethyl or propyl.

In another embodiment of the invention, in formula (I), R^{a} represents hydrogen and R^{b} represents hydrogen. In another embodiment, R^{a} represents hydrogen and R^{b} represents C₁₋₆-alkyl. In another embodiment, R^{a} represents C₁₋₆-alkyl and R^{b} represents hydrogen. In another embodiment, R^{a} represents C₁₋₆-alkyl and R^{b} represents C₁₋₆-alkyl.

In another embodiment of the invention, in formula (I), R^{c} represents phenyl. In another embodiment, R^{c} represents a monosubstituted phenyl. In another embodiment, R^{c} represents a disubstituted phenyl. In another embodiment, R^{c} represents a trisubstituted phenyl. In another embodiment, R^{c} represents a monohalo-substituted phenyl. In another embodiment, R^{c} represents a dihalo-substituted phenyl. In another embodiment, R^{c} represents a 3,4-dihalo-substituted phenyl. In another embodiment, R^{c} represents a trihalo-substituted phenyl. In another embodiment, R^{c} represents a monochloro-substituted phenyl e.g. 4-chlorophenyl. In another embodiment, R^{c} represents a dichloro-substituted phenyl, e.g. 3,4-dichlorophenyl. In another embodiment, R^{c} represents a dihalo-substituted phenyl, e.g. 4-bromo-3-chloro-phenyl. In another embodiment, R^{c} represents a trichloro-substituted phenyl, e.g 2,3,4-trichlorophenyl.

In another embodiment of the invention, in formula (I), R^{c} represents naphthyl. In another embodiment, R^{c} represents monosubstituted naphthyl. In another embodiment, R^{c} represents a disubstituted naphthyl. In another embodiment, R^{c} represents a trisubstituted naphthyl.

In another embodiment of the invention, in formula (I), R^{a} represents hydrogen, R^{b} represent C₁₋₆-alkyl, and R^{c} represents mono-halosubstituted phenyl.

In another embodiment of the invention, in formula (I), R^{a} represents hydrogen, R^{b} represent C₁₋₆-alkyl, and R^{c} represents di-halosubstituted phenyl.

In another embodiment of the invention, in formula (I), R^{a} represents hydrogen, R^{b} represent C₁₋₆-alkyl, and R^{c} represents tri-halosubstituted phenyl.

In another embodiment of the invention, in formula (I), R^{a} represents hydrogen, R^{b} represent C₁₋₆-alkyl, and R^{c} represents naphthyl.

In another embodiment of the invention, in formula (I), R^{a} and R^{b} represent hydrogen, and R^{c} represents a di-halosubstituted phenyl.

In another embodiment of the invention, in formula (I), R^{a} represents C₁₋₆-alkyl, R^{b} represent hydrogen, and R^{c} represents di-halosubstituted phenyl

In another embodiment of the invention, in formula (I), R^{a} and R^{b} represent C₁₋₆-alkyl, and R^{c} represents mono-halosubstituted phenyl.

In another embodiment of the invention, in formula (I), R^{a} and R^{b} represent C₁₋₆-alkyl, and R^{c} represents di-halosubstituted phenyl.

In another embodiment of the invention, in formula (I), R^{a} and R^{b} represent C₁₋₆-alkyl, and R^{c} represents tri-halosubstituted phenyl.

In another embodiment of the invention, the compound of the invention is:
N-(3,4-Dichloro-phenyl)-N-ethyl-3-piperidin-4-yl-propionamide;
N-(3,4-Dichloro-phenyl)-N-ethyl-3-(1-methyl-piperidin-4-yl)-propionamide; or
   a pharmaceutically acceptable salt thereof.

In another embodiment of the invention, the compound of the invention is:
N-(3,4-Dichloro-phenyl)-N-methyl-3-piperidin-4-yl-propionamide;
N-(3,4-Dichloro-phenyl)-3-piperidin-4-yl-N-propyl-propionamide;
N-(4-Bromo-3-chloro-phenyl)-N-ethyl-3-piperidin-4-yl-propionamide;
N-(3,4-Dichloro-phenyl)-N-methyl-3-(1-methyl-piperidin-4-yl)-propionamide;
3,4-Dichloro-phenyl)-3-(1-methyl-piperidin-4-yl)-N-propyl-propionamide;
N-(4-Bromo-3-chloro-phenyl)-N-ethyl-3-(1-methyl-piperidin-4-yl)-propionamide; or a pharmaceutically acceptable salt thereof.

Any combination of two or more of the embodiments as described above is considered within the scope of the present invention.

### Definition of Substituents

As used throughout the present specification and appended claims, the following terms have the indicated meaning:

The term “C₁₋₆-alkyl” as used herein means a saturated, branched or straight hydrocarbon group having from 1-6 carbon atoms, e.g. C₁₋₃-alkyl, C₁₋₄-alkyl, C₁₋₆-alkyl, C₂₋₆-alkyl, C₃₋₆-alkyl, and the like. Representative examples are methyl, ethyl, propyl (e.g. prop-1-yl, prop-2-yl (or iso-propyl)), butyl (e.g. 2-methylprop-2-yl (or tert-butyl), but-1-yl, but-2-yl), pentyl (e.g. pent-1-yl, pent-2-yl, pent-3-yl), 2-methylbut-1-yl, 3-methylbut-1-yl, hexyl (e.g. hex-1-yl), heptyl (e.g. hept-1-yl), octyl (e.g. oct-1-yl), nonyl (e.g. non-1-yl), and the like.

The term “halo” or “halogen” shall mean fluorine, chlorine, bromine or iodine.

The term “hydroxy” shall mean the radical —OH.

The term “cyano” shall mean the radical —CN.

The term “trihalomethyl” shall mean trifluoromethyl, trichloromethyl, and similar trihalo-substituted methyl groups.

The term “alkoxy” as used herein refers to the radical alkyl-O—. Representative examples are methoxy, ethoxy, propoxy (e.g. 1-propoxy, 2-propoxy), butoxy (e.g. 1-butoxy, 2-butoxy, 2-methyl-2-propoxy), pentoxy (1-pentoxy, 2-pentoxy), hexoxy (1-hexoxy, 3-hexoxy), and the like.

The term “trihalomethoxy” shall mean trifluoromethoxyl, trichloromethoxy, and similar trihalo-substituted methoxy groups.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

The terms “disease”, “condition” and “disorder” as used herein are used interchangeably to specify a state of a patient which is not the normal physiological state of man.

The term “medicament” as used herein means a pharmaceutical composition suitable for administration of the pharmaceutically active compound to a patient.

The term “pharmaceutically acceptable” as used herein means suited for normal pharmaceutical applications, i.e. giving rise to no adverse events in patients etc.

The term “effective amount” as used herein means a dosage which is sufficient in order for the treatment of the patient to be effective compared with no treatment.

The term “therapeutically effective amount” of a compound as used herein means an amount sufficient to cure, alleviate or partially arrest the clinical manifestations of a given disease and its complications. An amount adequate to accomplish this is defined as “therapeutically effective amount”. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

### Pharmaceutically Acceptable Salts

The chemical compound of the invention may be provided in any form suitable for the intended administration. Suitable forms include pharmaceutically (i.e. physiologically) acceptable salts, and pre- or prodrug forms of the chemical compound of the invention.

Examples of pharmaceutically acceptable addition salts include, without limitation, the non-toxic inorganic and organic acid addition salts such as the hydrochloride, the hydrobromide, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

Examples of pharmaceutically acceptable cationic salts of a chemical compound of the invention include, without limitation, the sodium, the potassium, the calcium, the magnesium, the zinc, the aluminium, the lithium, the choline, the lysinium, and the ammonium salt, and the like, of a chemical compound of the invention containing an anionic group. Such cationic salts may be formed by procedures well known and described in the art.

In the context of this invention the “onium salts” of N-containing compounds are also contemplated as pharmaceutically acceptable salts. Preferred “onium salts” include the alkyl-onium salts, the cycloalkyl-onium salts, and the cycloalkylalkyl-onium salts.

Examples of pre- or prodrug forms of the chemical compound of the invention include examples of suitable prodrugs of the substances according to the invention include compounds modified at one or more reactive or derivatizable groups of the parent compound. Of particular interest are compounds modified at a carboxyl group, a hydroxyl group, or an amino group. Examples of suitable derivatives are esters or amides.

The chemical compound of the invention may be provided in dissoluble or indissoluble forms together with a pharmaceutically acceptable solvent such as water, ethanol, and the like. Dissoluble forms may also include hydrated forms such as the monohydrate, the dihydrate, the hemihydrate, the trihydrate, the tetrahydrate, and the like. In general, the dissoluble forms are considered equivalent to indissoluble forms for the purposes of this invention.

Steric Isomers

It will be appreciated by those skilled in the art that the compounds of the present invention may exist in different stereoisomeric forms—including enantiomers, diastereomers or cis-trans-isomers.

The invention includes all such isomers and any mixtures thereof including racemic mixtures.

Racemic forms can be resolved into the optical antipodes by known methods and techniques. One way of separating the enantiomeric compounds (including enantiomeric intermediates) is—in the case the compound being a chiral acid—by use of an optically active amine, and liberating the diastereomeric, resolved salt by treatment with an acid. Another method for resolving racemates into the optical antipodes is based upon chromatography on an optical active matrix. Racemic compounds of the present invention can thus be resolved into their optical antipodes, e.g., by fractional crystallisation of D- or L- (tartrates, mandelates, or camphorsulphonate) salts for example.

The chemical compounds of the present invention may also be resolved by the formation of diastereomeric amides by reaction of the chemical compounds of the present invention with an optically active activated carboxylic acid such as that derived from (+) or (−) phenylalanine, (+) or (−) phenylglycine, (+) or (−) camphanic acid or by the formation of diastereomeric carbamates by reaction of the chemical compound of the present invention with an optically active chloroformate or the like.

Additional methods for the resolving the optical isomers are known in the art. Such methods include those described by Jaques J, Collet A, & Wilen S in “Enantiomers, Racemates, and Resolutions”, John Wiley and Sons, New York (1981).

Optical active compounds can also be prepared from optical active starting materials.

### Labelled Compounds

The compounds of the invention may be used in their labelled or unlabelled form. In the context of this invention the labelled compound has one or more atoms replaced by an atom having an atomic mass or mass number different from the atomic mass or mass number usually found in nature. The labelling will allow easy quantitative detection of said compound.

The labelled compounds of the invention may be useful as diagnostic tools, radio tracers, or monitoring agents in various diagnostic methods, and for in vivo receptor imaging.

The labelled isomer of the invention preferably contains at least one radionuclide as a label. Positron emitting radionuclides are all candidates for usage. In the context of this invention the radionuclide is preferably selected from ²H (deuterium), ³H (tritium), ¹¹C, ¹³C, ¹⁴C, ¹³¹I, ¹²⁵I, ¹²³I, and ¹⁸F.

The physical method for detecting the labelled isomer of the present invention may be selected from Position Emission Tomography (PET), Single Photon Imaging Computed Tomography (SPECT), Magnetic Resonance Spectroscopy (MRS), Magnetic Resonance Imaging (MRI), and Computed Axial X-ray Tomography (CAT), or combinations thereof.

### Methods of Preparation

The chemical compounds of the invention may be prepared by conventional methods for chemical synthesis, e.g. those described in the working examples. The starting materials for the processes described in the present application are known or may readily be prepared by conventional methods from commercially available chemicals.

Also one compound of the invention can be converted to another compound of the invention using conventional methods.

The end products of the reactions described herein may be isolated by conventional techniques, e.g. by extraction, crystallisation, distillation, chromatography, etc.

### Biological Activity

Compounds of the invention may be tested for their ability to inhibit reuptake of the monoamines dopamine, noradrenaline and serotonin in synaptosomes e.g. such as described in WO 97/30997 (NeuroSearch A/S). Based on the balanced activity observed in these tests the compound of the invention is considered useful for the treatment, prevention or alleviation of a disease or a disorder or a condition of a mammal, including a human, which disease, disorder or condition is responsive to inhibition of monoamine neurotransmitter re-uptake in the central nervous system.

In a special embodiment, the compounds of the invention are considered useful for the treatment, prevention or alleviation of: mood disorder, depression, atypical depression, depression secondary to pain, major depressive disorder, dysthymic disorder, bipolar disorder, bipolar I disorder, bipolar II disorder, cyclothymic disorder, mood disorder due to a general medical condition, substance-induced mood disorder, pseudodementia, Ganser's syndrome, obsessive compulsive disorder, panic disorder, panic disorder without agoraphobia, panic disorder with agoraphobia, agoraphobia without history of panic disorder, panic attack, memory deficits, memory loss, attention deficit hyperactivity disorder, obesity, anxiety, generalized anxiety disorder, eating disorder, Parkinson's disease, parkinsonism, dementia, dementia of ageing, senile dementia, Alzheimer's disease, Down's syndrome, acquired immunodeficiency syndrome dementia complex, memory dysfunction in ageing, specific phobia, social phobia, social anxiety disorder, post-traumatic stress disorder, acute stress disorder, drug addiction, drug abuse, drug abuse liability, cocaine abuse, nicotine abuse, tobacco abuse, alcohol addiction, alcoholism, kleptomania, withdrawal symptoms caused by termination of use of addictive substances, pain, chronic pain, inflammatory pain, neuropathic pain, migraine pain, tension-type headache, chronic tension-type headache, pain associated with depression, fibromyalgia, arthritis, osteoarthritis, rheumatoid arthritis, back pain, cancer pain, irritable bowel pain, irritable bowel syndrome, post-operative pain, post-mastectomy pain syndrome (PMPS), post-stroke pain, drug-induced neuropathy, diabetic neuropathy, sympathetically-maintained pain, trigeminal neuralgia, dental pain, myofacial pain, phantom-limb pain, bulimia, premenstrual syndrome, premenstrual dysphoric disorder, late luteal phase syndrome, post-traumatic syndrome, chronic fatigue syndrome, persistent vegetative state, urinary incontinence, stress incontinence, urge incontinence, nocturnal incontinence, sexual dysfunction, premature ejaculation, erectile difficulty, erectile dysfunction, premature female orgasm, restless leg syndrome, periodic limb movement disorder, eating disorders, anorexia nervosa, sleep disorders, pervasive developmental disorders, autism, Asperger's disorder, Rett's disorder, childhood disintegrative disorder, learning disabilities, motor skills disorders, mutism, trichotillomania, narcolepsy, post-stroke depression, stroke-induced brain damage, stroke-induced neuronal damage, Gilles de la Tourettes disease, tinnitus, tic disorders, body dysmorphic disorders, oppositional defiant disorder or post-stroke disabilities. In a preferred embodiment, the compounds are considered useful for the treatment, prevention or alleviation of depression.

It is at present contemplated that a suitable dosage of the active pharmaceutical ingredient (API) is within the range of from about 0.1 to about 1000 mg API per day, more preferred of from about 10 to about 500 mg API per day, most preferred of from about 30 to about 100 mg API per day, dependent, however, upon the exact mode of administration, the form in which it is administered, the indication considered, the subject and in particular the body weight of the subject involved, and further the preference and experience of the physician or veterinarian in charge.

Preferred compounds of the invention show a biological activity in the submicromolar and micromolar range, i.e. of from below 1 to about 100 μM.

### Pharmaceutical Compositions

In another aspect the invention provides novel pharmaceutical compositions comprising a therapeutically effective amount of the chemical compound of the invention.

While a chemical compound of the invention for use in therapy may be administered in the form of the raw chemical compound, it is preferred to introduce the active ingredient, optionally in the form of a physiologically acceptable salt, in a pharmaceutical composition together with one or more adjuvants, excipients, carriers, buffers, diluents, and/or other customary pharmaceutical auxiliaries.

In a preferred embodiment, the invention provides pharmaceutical compositions comprising the chemical compound of the invention, or a pharmaceutically acceptable salt or derivative thereof, together with one or more pharmaceutically acceptable carriers, and, optionally, other therapeutic and/or prophylactic ingredients, known and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in dragé, in powder, or in liquid form, and parenteral administration, in particular cutaneous, subcutaneous, intramuscular, or intravenous injection. The pharmaceutical composition of the invention can be manufactured by any skilled person by use of standard methods and conventional techniques appropriate to the desired formulation. When desired, compositions adapted to give sustained release of the active ingredient may be employed.

Further details on techniques for formulation and administration may be found in the latest edition of Remington's Pharmaceutical Sciences (Maack Publishing Co., Easton, Pa.).

The actual dosage depends on the nature and severity of the disease being treated, and is within the discretion of the physician, and may be varied by titration of the dosage to the particular circumstances of this invention to produce the desired therapeutic effect. However, it is presently contemplated that pharmaceutical compositions containing of from about 0.1 to about 500 mg of active ingredient per individual dose, preferably of from about 1 to about 100 mg, most preferred of from about 1 to about 10 mg, are suitable for therapeutic treatments.

The active ingredient may be administered in one or several doses per day. A satisfactory result can, in certain instances, be obtained at a dosage as low as 0.1 μg/kg i.v. and 1 μg/kg p.o. The upper limit of the dosage range is presently considered to be about 10 mg/kg i.v. and 100 mg/kg p.o. Preferred ranges are from about 0.1 μg/kg to about 10 mg/kg/day i.v., and from about 1 μg/kg to about 100 mg/kg/day p.o.

### Methods of Therapy

In another aspect the invention provides a method for the treatment, prevention or alleviation of a disease or a disorder or a condition of a living animal body, including a human, which disease, disorder or condition is responsive to inhibition of monoamine neurotransmitter re-uptake in the central nervous system, and which method comprises administering to such a living animal body, including a human, in need thereof an effective amount of a chemical compound of the invention.

It is at present contemplated that suitable dosage ranges are 0.1 to 1000 milligrams daily, 10-500 milligrams daily, and especially 30-100 milligrams daily, dependent as usual upon the exact mode of administration, form in which administered, the indication toward which the administration is directed, the subject involved and the body weight of the subject involved, and further the preference and experience of the physician or veterinarian in charge.

### EXAMPLES

The following examples and general procedures refer to intermediate compounds and final products for general formula (I) identified in the specification. The preparation of the compounds of general formula (I) of the present invention is described in detail using the following examples. Occasionally, the reaction may not be applicable as described to each compound included within the disclosed scope of the invention. The compounds for which this occurs will be readily recognized by those skilled in the art. In these cases the reactions can be successfully performed by conventional modifications known to those skilled in the art, which is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials.

All reactions involving air sensitive reagents or intermediates are performed under nitrogen and in anhydrous solvents. Magnesium sulphate is used as drying agent in the workup-procedures and solvents are evaporated under reduced pressure.

The abbreviations as used in the examples have the following meaning:
TLC: Thin layer chromatography
CDCl₃: Deuterio chloroform

### DCM: Dichloromethane

### DIIC: N,N′-Diisopropylcarbodiimide

### DMAP: 4-Dimethylaminopyridine

DMSO-Cl₆: Hexadeuterio dimethylsulfoxide

### DMSO: Dimethylsulfoxide

### DIPEA: Diisopropylethylamine

EDAC: 1-(3-Dimethylaminopropyl)-3-ethylcarbodiimide hydrochloride
EtOAc: Ethyl acetate

### THF: Tetrahydrofuran

### DMF: N,N-dimethylformamide

### HOBT: 1-Hydroxy-benzotriazole

### POL: Polystyrene

### MeCN: Acetonitrile

### NMP: N-Methylpyrrolidinone

### TEA: Triethylamine

TFA: Trifluoroacetic acid
EDAC: 1-(3-Dimethylaminopropyl)-3-ethylcarbodiimide, hydrochloride
min: minutes
hrs: hours

### Example 1

### 4-[2-(3,4-Dichloro-phenylcarbamoyl)-ethyl]-piperidine-1-carboxylic acid tert-butyl ester (Intermediate)

A mixture of 3,4-dichloroaniline (0.32 g, 1.94 mmol), 1-boc-piperidine-4-yl-propionic acid (0.50 g, 1.94 mmol), N-ethyl-N″-(3-dimethylaminopropyl)carbodiimide×HCl (EDCI), acetic acid (0.29 g, 2.14 mmol) and dichloromethane (7 ml). Water and aqueous sodium hydrogen carbonate were added. The mixture was extracted with dichloromethane, dried and evaporated. Yield 0.68 g (87%).

### Example 2

### 4-{2-[(3,4-Dichloro-phenyl)-ethyl-carbamoyl]-ethyl}-piperidine-1-carboxylic acid tert-butyl ester (Intermediate)

4-[2-(3,4-Dichloro-phenylcarbamoyl)-ethyl]-piperidine-1-carboxylic acid tert-butyl ester (0.68 g, 1.69 mmol) solved in THF (5 ml) was added dropwise to a mixture of sodium hydride (60%, 0.081 g, 2.03 mmol) and THF (5 ml). The reaction-mixture was stirred for 30 min at room temperature. Ethyliodide (0.32 g, 2.03 mmol) was added and the reaction-mixture was stirred at room-temperature for 15 h. Aqueous sodium hydrogenecarbonate was added and extracted with dichloromethane, dried and evaporated. The crude product was purified by column silica gel chromatography by using a mixture of ethylacetate 5-55% and heptane as solvent. The product was isolated as an oil. Yield 547 mg (75%).

### Example 3

### N-(3,4-Dichloro-phenyl)-N-ethyl-3-piperidin-4-yl-propionamide fumaric acid salt (Compound 3.1)

A mixture of 4-{2-[(3,4-dichloro-phenyl)-ethyl-carbamoyl]-ethyl}-piperidine-1-carboxylic acid tert-butyl ester (0.547 g, 1.27 mmol) trifluoroacetic acid (1.0 ml, 13.46 mmol) was stirred at room-temperature for 3 h. The mixture was coevaporated with toluene. The free base was obtained using ion exchange (SCX). The corresponding fumaric acid salt was prepared by dissolving the product in a mixture of MeOH and dichloromethane (1:1). One equivalent of fumaric acid was added and the clear solution was evaporated. Yield 374 mg (100%). Mp 160-162° C.

LC-ESI-HRMS of [M+H]+ shows 329.1181 Da. Calc. 329.118199 Da, dev. −0.3 ppm.

### N-(3,4-Dichloro-phenyl)-N-methyl-3-piperidin-4-yl-propionamide fumaric acid salt (Compound 3.2)

Was prepared according to example 3.

LC-ESI-HRMS of [M+H]+ shows 315.1029 Da. Calc. 315.102549 Da, dev. 1.1 ppm.

### N-(3,4-Dichloro-phenyl)-3-piperidin-4-yl-N-propyl-propionamide fumaric acid salt (Compound 3.3)

Was prepared according to example 3.

LC-ESI-HRMS of [M+H]+ shows 343.1354 Da. Calc. 343.133849 Da, dev. 4.5 ppm.

### N-(4-Bromo-3-chloro-phenyl)-N-ethyl-3-piperidin-4-yl-propionamide fumaric acid salt (Compound 3.4)

Was prepared according to example 3.

LC-ESI-HRMS of [M+H]+ shows 373.0691 Da. Calc. 373.067684 Da, dev. 3.8 ppm.

### Example 4

### N-(3,4-Dichloro-phenyl)-N-ethyl-3-(1-methyl-piperidin-4-yl)-propionamide fumaric acid salt (Compound 4.1)

N-(3,4-Dichloro-phenyl)-N-ethyl-3-piperidin-4-yl-propionamide (0.15 g, 0.45 mmol) was dissolved in dichloromethane (3 ml). Formaldehyde (37% solution in water) (0.041 ml, 1.368 mmol) was added followed by sodium triacetoxyborohydride (0.398 g, 1.82 mmol). The mixture was stirred at room-temperature for 20 h. Aqueous saturated sodium hydrogenecarbonate was added and the mixture was extracted with dichloromethane, using a water-separator. The free base was isolated as colourless oil. Yield 73 mg (46%). The corresponding fumaric acid salt was prepared by dissolving the product in a mixture of MeOH and dichloromethane (1:1). One equivalent of fumaric acid was added and the clear solution was evaporated. Yield 95 mg (97%). LC-ESI-HRMS of [M+H]+ shows 343.1345 Da. Calc. 343.133849 Da, dev. 1.9 ppm.

### N-(3,4-Dichloro-phenyl)-N-methyl-3-(1-methyl-piperidin-4-yl)-propionamide fumaric acid salt (Compound 4.2)

Was prepared according to example 4.

LC-ESI-HRMS of [M+H]+ shows 329.1190456 Da. Calc. 329.118199 Da, dev. 2.6 ppm.

### N-(3,4-Dichloro-phenyl)-3-(1-methyl-piperidin-4-yl)-N-propyl-propionamide fumaric acid salt (Compound 4.3)

Was prepared according to example 4.

LC-ESI-HRMS of [M+H]+ shows 357.1506733 Da. Calc. 357.149499 Da, dev. 3.3 ppm.

### N-(4-Bromo-3-chloro-phenyl)-N-ethyl-3-(1-methyl-piperidin-4-yl)-propionamide fumaric acid salt (Compound 4.4)

Was prepared according to example 4.

LC-ESI-HRMS of [M+H]+ shows 387.0836 Da. Calc. 387.083334 Da, dev. 0.7 ppm.

### In Vitro Inhibition Activity

Compounds were tested for their ability to inhibit the reuptake of the monoamine neurotransmitters dopamine (DA) noradrenaline (NA) and serotonine (5-HT) in synaptosomes as described in WO 97/16451 (NeuroSearch A/S).

The test values are given as IC₅₀ (the concentration (μM) of the test substance which inhibits the specific binding of ³H-DA, ³H-NA, or ³H-5-HT by 50%).

Test results obtained by testing compounds of the present invention appear from the below table:

TABLE 1

| | 5-HT- | DA- | NA- |
|---|---|---|---|
| | uptake | uptake | uptake |
| | IC₅₀ | IC₅₀ | IC₅₀ |
| Test compound | (μM) | (μM) | (μM) |
| N-(3,4-Dichloro-phenyl)-N-ethyl-3- | 2.1 | 0.011 | 0.011 |
| piperidin-4-yl-propionamide | | | |
| N-(3,4-Dichloro-phenyl)-N-methyl-3- | >1.0 | 0.22 | 0.77 |
| piperidin-4-yl-propionamide | | | |
| N-(3,4-Dichloro-phenyl)-3-piperidin-4- | 1.4 | 0.0098 | 0.0019 |
| yl-N-propyl-propionamide | | | |
| N-(4-Bromo-3-chloro-phenyl)-N-ethyl- | >1.0 | 0.0031 | 0.0026 |
| 3-piperidin-4-yl-propionamide | | | |
| N-(3,4-Dichloro-phenyl)-N-ethyl-3-(1- | >1.0 | 0.0063 | 0.0065 |
| methyl-piperidin-4-yl)-propionamide | | | |
| N-(3,4-Dichloro-phenyl)-N-methyl-3-(1- | >1.0 | 0.33 | 0.087 |
| methyl-piperidin-4-yl)-propionamide | | | |
| 3,4-Dichloro-phenyl)-3-(1-methyl- | 0.82 | 0.010 | 0.0010 |
| piperidin-4-yl)-N-propyl-propionamide | | | |
| N-(4-Bromo-3-chloro-phenyl)-N-ethyl- | >1.0 | 0.0068 | 0.0013 |
| 3-(1-methyl-piperidin-4-yl)- | | | |
| propionamide | | | |

From the foregoing it will be appreciated that, although specific embodiments of the invention have been described herein for purposes of illustration, various modifications may be made without deviating from the spirit and scope of the invention. Accordingly, the invention is not to be limited as by the appended claims.

1. A compound of Formula (I): 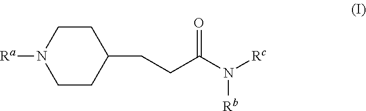
any of its stereoisomers or any mixture of its stereoisomers, or a pharmaceutically acceptable salt thereof, wherein
R^{a} and R^{b}, independently of each other, represent hydrogen or C₁₋₆-alkyl; and
R^{c} represents phenyl or naphthyl, which phenyl and naphtyl are optionally substituted with one or more substituents independently selected from the group consisting of halo, trifluoromethyl, trifluoromethoxy, cyano and alkoxy.

2. The compound according to claim 1, any of its stereoisomers or any mixture of its stereoisomers, or a pharmaceutically acceptable salt thereof, wherein R^{a} represents hydrogen.

3. The compound according to claim 1, any of its stereoisomers or any mixture of its stereoisomers, or a pharmaceutically acceptable salt thereof, wherein R^{a} represents C₁₋₆-alkyl.

4. The compound according to claim 1, any of its stereoisomers or any mixture of its stereoisomers, or a pharmaceutically acceptable salt thereof, wherein R^{b} represents C₁₋₆-alkyl.

5. The compound according to claim 1, any of its stereoisomers or any mixture of its stereoisomers, or a pharmaceutically acceptable salt thereof, wherein R^{c} represents dihalophenyl.

6. The compound according to claim 1, which is
N-(3,4-Dichloro-phenyl)-N-ethyl-3-piperidin-4-yl-propionamide;
N-(3,4-Dichloro-phenyl)-N-ethyl-3-(1-methyl-piperidin-4-yl)-propionamide; or any of its stereoisomers or any mixture of its stereoisomers, or a pharmaceutically acceptable salt thereof.

7. The compound according to claim 1, which is
N-(3,4-Dichloro-phenyl)-N-methyl-3-piperidin-4-yl-propionamide;
N-(3,4-Dichloro-phenyl)-3-piperidin-4-yl-N-propyl-propionamide;
N-(4-Bromo-3-chloro-phenyl)-N-ethyl-3-piperidin-4-yl-propionamide;
N-(3,4-Dichloro-phenyl)-N-methyl-3-(1-methyl-piperidin-4-yl)-propionamide;
3,4-Dichloro-phenyl)-3-(1-methyl-piperidin-4-yl)-N-propyl-propionamide;
N-(4-Bromo-3-chloro-phenyl)-N-ethyl-3-(1-methyl-piperidin-4-yl)-propionamide;
or any of its stereoisomers or any mixture of its stereoisomers, or a pharmaceutically acceptable salt thereof.

8. A pharmaceutical composition, comprising a therapeutically effective amount of a compound of claim 1, any of its stereoisomers or any mixture of its stereoisomers, or a pharmaceutically acceptable salt thereof, together with at least one pharmaceutically acceptable carrier, excipient or diluent.

9. (canceled)

10. (canceled)

11. (canceled)

12. A method for treatment, prevention or alleviation of a disease or a disorder or a condition of a living animal body, including a human, which disorder, disease or condition is responsive to inhibition of monoamine neurotransmitter re-uptake in the central nervous system, which method comprises the step of administering to such a living animal body in need thereof a therapeutically effective amount of a compound according to claim 1, or any of its stereoisomers or any mixture of its stereoisomers, or a pharmaceutically acceptable salt thereof.

13. A compound according to claim 1, any of its stereoisomers or any mixture of its stereoisomers, or a pharmaceutically acceptable salt thereof, for use as a medicament.

14. A compound according to claim 1, any of its stereoisomers or any mixture of its stereoisomers, or a pharmaceutically acceptable salt thereof, for use in the treatment, prevention or alleviation of a disease or a disorder or a condition of a mammal, including a human, which disease, disorder or condition is responsive to inhibition of monoamine neurotransmitter re-uptake in the central nervous system.

15. The method according to claim 12, wherein the disease, disorder or condition is mood disorder, depression, atypical depression, depression secondary to pain, major depressive disorder, dysthymic disorder, bipolar disorder, bipolar I disorder, bipolar II disorder, cyclothymic disorder, mood disorder due to a general medical condition, substance-induced mood disorder, pseudodementia, Ganser's syndrome, obsessive compulsive disorder, panic disorder, panic disorder without agoraphobia, panic disorder with agoraphobia, agoraphobia without history of panic disorder, panic attack, memory deficits, memory loss, attention deficit hyperactivity disorder, obesity, anxiety, generalized anxiety disorder, eating disorder, Parkinson's disease, parkinsonism, dementia, dementia of ageing, senile dementia, Alzheimer's disease, Down's syndrome, acquired immunodeficiency syndrome dementia complex, memory dysfunction in ageing, specific phobia, social phobia, social anxiety disorder, post-traumatic stress disorder, acute stress disorder, drug addiction, drug abuse, drug abuse liability, cocaine abuse, nicotine abuse, tobacco abuse, alcohol addiction, alcoholism, kleptomania, withdrawal symptoms caused by termination of use of addictive substances, pain, chronic pain, inflammatory pain, neuropathic pain, migraine pain, tension-type headache, chronic tension-type headache, pain associated with depression, fibromyalgia, arthritis, osteoarthritis, rheumatoid arthritis, back pain, cancer pain, irritable bowel pain, irritable bowel syndrome, post-operative pain, post-mastectomy pain syndrome (PMPS), post-stroke pain, drug-induced neuropathy, diabetic neuropathy, sympathetically-maintained pain, trigeminal neuralgia, dental pain, myofacial pain, phantom-limb pain, bulimia, premenstrual syndrome, premenstrual dysphoric disorder, late luteal phase syndrome, post-traumatic syndrome, chronic fatigue syndrome, persistent vegetative state, urinary incontinence, stress incontinence, urge incontinence, nocturnal incontinence, sexual dysfunction, premature ejaculation, erectile difficulty, erectile dysfunction, premature female orgasm, restless leg syndrome, periodic limb movement disorder, eating disorders, anorexia nervosa, sleep disorders, pervasive developmental disorders, autism, Asperger's disorder, Rett's disorder, childhood disintegrative disorder, learning disabilities, motor skills disorders, mutism, trichotillomania, narcolepsy, post-stroke depression, stroke-induced brain damage, stroke-induced neuronal damage, Gilles de la Tourettes disease, tinnitus, tic disorders, body dysmorphic disorders, oppositional defiant disorder or post-stroke disabilities.
